(19) United States
(12) Patent Application Publication
(10) Pub. No.: US 2017/0360378 A1
(43) Pub. Date: Dec. 21, 2017

(54) SYSTEMS AND METHODS FOR IDENTIFYING FETAL MOVEMENTS IN AN AUDIO/VISUAL DATA FEED AND USING THE SAME TO ASSESS FETAL WELL-BEING

(71) Applicants: Ghassan S. Kassab, La Jolla, CA (US); Sonky Ung, San Diego, CA (US)

(72) Inventors: Ghassan S. Kassab, La Jolla, CA (US); Sonky Ung, San Diego, CA (US)

(21) Appl. No.: 15/625,175

(22) Filed: Jun. 16, 2017

Related U.S. Application Data
(60) Provisional application No. 62/351,039, filed on Jun. 16, 2016.

(51) Int. Cl. A61B 5/00; A61B 5/11; A61B 7/04

(52) U.S. Cl. CPC A61B 5/7271; A61B 7/04; A61B 5/0082; A61B 5/1128; A61B 5/4362

(57) ABSTRACT

There is provided a system and methods for quantitatively assessing fetal well-being based on observing fetal movement activity in an audio/visual data feed. The system includes a method that detects and quantifies fetal movements in an audio/visual data feed by audio/visual-processing motion estimation techniques. The system also includes a method that captures metrics relating to fetal movements sensed by the mother or other independent party, whereby termed “maternal perception”. The system further includes a method that cross validates the fetal movement detected by the system with fetal movement sensed by “maternal perception”. The system further includes a method that generates output to summarize fetal movement activity over a recorded time period. This output may be reviewed by a third party to assist in determining if further intervention is needed.

### PRIORITY

The present application is related to, and claims the priority benefit of, U.S. Provisional Patent Application Ser. No. 62/351,039, filed Jun. 16, 2016, the contents of which are expressly incorporated herein directly and by reference in their entirety.

### FIELD

The present disclosure relates to perinatal monitoring and more particularly to detecting fetal movements in an audio/visual data feed and use of same for assessing fetal well-being, particularly by monitoring for abrupt changes in observed fetal movement activity.

### BACKGROUND

Among women who have delivered a live-born baby, more than 99% agreed with the statement that it was important to them to feel the baby move every day (Froen et al, “Fetal Movement Assessment,” Seminars in Perinatology, 32:243-346, 2008). Unsurprisingly, fetal movement as a sign of fetal well-being has received much attention in the literature over the past several decades. Of clinical concern is/are decreased fetal movements (DFM), because the incidence of adverse outcomes in pregnancies with DFM is significant (Froen et al., “Management of Decreased Fetal Movements,” Seminars in Perinatology, 32:307-311, 2008). Women presented with DFM are at increased risk of perinatal complications, specifically, stillbirth.

Fetal movement counting (FMC) can be used as an initial screening method in predicting fetal health (Kamalifard et al., “Diagnostic Value of Fetal Movement Counting by Mother and the Optimal Recording Duration,” Journal of Caring Sciences, 2(2):89-95, 2013). Pregnant women are encouraged to engage in fetal movement counting (FMC) as a way to self-screen for DFM (Kuwata et al, “Establishing a reference value for the frequency of fetal movements using modified ‘count to 10’ method,” Japan Society of Obstetrics and Gynecology Research, 34(3):318-323, 2008). Although several protocols have been used to count fetal movements, neither the optimal number of movements nor the ideal duration for counting them has been defined. The definition of DFM is therefore not universal (Velazquez and Rayburn, “Antenatal Evaluation of the Fetus Using Fetal Movement Monitoring,” Clinical Obstetrics and Gynecology, 45(4):993-1004, 2002).

In addition to counting fetal movements, movements can be further distinguished based on strength and speed of the whole body or limb-only fetal movements. Maternal perception of the intensity and duration of the movements can give additional information about the unborn baby's fitness (Radestad, “Strengthening Mindfetalness,” Sexual & Reproductive Healthcare, 3:59-60, 2012). Maternally perceived fetal movements, however, are subjective by nature due to the sensitivity of the mother to recognize these fetal movements. The small number of women who are incapable of recording perceived fetal movement often improve their perceptive ability when viewing activity during real-time ultrasound examinations (Velazquez and Rayburn, 2002). To learn to develop self-confidence in perceiving fetal movement and begin to rely on what one feels takes time (Radestad, 2012).

### BRIEF SUMMARY

Pregnant women might encounter specific situations in which it is desirable to record the movement activity of their fetus. Such recording should quantify the movement activity of their fetus, and this quantified movement activity should correlate well with maternally perceived fetal movement activity for validation purposes. In particular, when irregular fetal movement activity is maternally perceived it may be desirable for a third party to assess the recorded fetal movement activity and determine whether further intervention is needed.

What would be desirable therefore is an automated system that can objectively recognize and quantify decreased fetal movements as a precursor to high-risk perinatal cases that require further intervention.

In an exemplary embodiment of a system of the present disclosure, the system comprises one or more of the following components and/or devices: one or more microphones configured to obtain fetal movement data san an input audio data stream used by one or more system components; and/or one or more visual (such as video) cameras operating in the visible light, infrared, and/or radio frequency spectrum configured to obtain fetal movement data as an input visual data stream used by one or more system components; one or more audio/visual recording devices configured to obtain fetal movement data as an input audio and visual/video data stream used by one or more system components; and/or one or more motion processing engines configured to receive data, process the same to generate motion detection data, and/or transmit said motion detection data; and/or one or more analytics engines configured to receive data, process the same to generate analyzed motion data, and/or transmit said analyzed motion data; and/or one or more data depots configured to receive data, store said data, and/or transmit said data; and/or one or more validate components configured to receive data, process the same to generate validated data, and/or transmit said validated data; and/or one or more user interaction modules configured to receive data, display data, and/or process said data, and/or transmit said data. In an exemplary embodiment of a system of the present disclosure, the system is configured as one device, two devices, three devices, or more than three devices. In an exemplary embodiment of a system of the present disclosure, the system is configured to generate data to determine fetal well-being. In an exemplary embodiment of a system of the present disclosure, data generated by the system can be used to diagnose a fetal condition.

The present disclosure includes disclosure of a system, as shown and/or described herein. The present disclosure also includes disclosure of a method of using a system, as shown and/or described herein.

The present disclosure includes disclosure of an exemplary system, comprising a recording device configured to obtain audio and/or visual data when directed toward a womb of a pregnant woman and further configured to transmit the audio and/or visual data; a motion processing engine configured to receive the audio and/or visual data, detect motion within the audio and/or visual data, and generate motion data based upon the motion detected within the audio and/or visual data; an analytics engine configured to receive the motion data from the motion processing engine, analyze the motion data to determine of the motion data corresponds to a one or more of fetal motion, maternal motion, or other motion, and generate analyzed motion data corresponding to the one or more of fetal motion, maternal motion, or other motion; and a data depot configured to receive the analyzed motion data from the analytics engine, store the same as stored data, and transmit the stored data.

The present disclosure includes disclosure of an exemplary system, wherein the analytics engine is further configured to receive the audio and/or visual data from the recording device.

The present disclosure includes disclosure of an exemplary system, wherein the data depot is further configured to receive the audio and/or visual data from the recording device and to store the same as stored data.

The present disclosure includes disclosure of an exemplary system, further comprising a user interaction module configured to receive analyzed motion data from the analytics engine, to receive maternal perception input data synchronized with the audio and/or visual data, and to display at least one of the analyzed motion data and/or the maternal perception input data.

The present disclosure includes disclosure of an exemplary system, wherein the user interaction module is an input/output device.

The present disclosure includes disclosure of an exemplary system, further comprising a validate component configured to receive the stored data from the data depot, compare the stored data with the maternal perception input data to generate validated data, and to transmit the validated data to the data depot to be stored as additional stored data.

The present disclosure includes disclosure of an exemplary system, configured for operation upon a single device, the single device comprising an input/output interface; a processor/memory storage/network interface; and the recording device.

The present disclosure includes disclosure of an exemplary system, wherein the single device is selected from the group consisting of a smartphone, a tablet computer, and a laptop computer.

The present disclosure includes disclosure of an exemplary system, configured for operation upon a first device and a second device, the first device comprising an input/output interface and a processor/memory storage/network interface, and the second device comprising the recording device, wherein the first device is configured to receive the audio and/or visual data from the second device.

The present disclosure includes disclosure of an exemplary system, wherein the first device is selected from the group consisting of a smartphone, a tablet computer, and a laptop computer.

The present disclosure includes disclosure of an exemplary system, configured for operation upon a first device, a second device, and a third device, the first device comprising an input/output interface, the second device comprising the recording device, and the third device comprising a processor/memory storage/network interface.

The present disclosure includes disclosure of an exemplary system, wherein the third device is configured to receive the audio and/or visual data from the second device, store the audio and/or visual data as the stored data, and transmit the stored data to the first device.

The present disclosure includes disclosure of an exemplary system, wherein the first device comprises a smartphone or a tablet, and wherein the third device comprises a laptop computer or a desktop computer.

The present disclosure includes disclosure of an exemplary system, wherein the stored data is indicative of fetal well-being.

The present disclosure includes disclosure of an exemplary system, wherein the stored data is indicative of a diagnosis of a fetal condition.

The present disclosure includes disclosure of an exemplary system, comprising a recording device configured to obtain audio and/or visual data when directed toward a womb of a pregnant woman and further configured to transmit the audio and/or visual data; a motion processing engine configured to receive the audio and/or visual data, detect motion within the audio and/or visual data, and generate motion data based upon the motion detected within the audio and/or visual data; an analytics engine configured to receive the motion data from the motion processing engine, analyze the motion data to determine of the motion data corresponds to a one or more of fetal motion, maternal motion, or other motion, and generate analyzed motion data corresponding to the one or more of fetal motion, maternal motion, or other motion; a data depot configured to receive the analyzed motion data from the analytics engine, store the same as stored data, and transmit the stored data; and a validate component configured to receive the stored data from the data depot, compare the stored data with maternal perception input data obtained by a user interaction module to generate validated data, and to transmit the validated data to the data depot to be stored as additional stored data.

The present disclosure includes disclosure of an exemplary system, configured for operation upon a single device, the single device comprising an input/output interface; a processor/memory storage/network interface; and the recording device; wherein the single device is selected from the group consisting of a smartphone, a tablet computer, and a laptop computer.

The present disclosure includes disclosure of an exemplary system, configured for operation upon a first device and a second device, the first device comprising an input/output interface and a processor/memory storage/network interface, and the second device comprising the recording device, wherein the first device is configured to receive the audio and/or visual data from the second device, and wherein the first device is selected from the group consisting of a smartphone, a tablet computer, and a laptop computer.

The present disclosure includes disclosure of an exemplary system, configured for operation upon a first device, a second device, and a third device, the first device comprising an input/output interface, the second device comprising the recording device, and the third device comprising a processor/memory storage/network interface, wherein the third device is configured to receive the audio and/or visual data from the second device, store the audio and/or visual data as the stored data, and transmit the stored data to the first device, and wherein the first device comprises a smartphone or a tablet, and wherein the third device comprises a laptop computer or a desktop computer.

The present disclosure includes disclosure of an exemplary method of determining fetal well-being, comprising the steps of operating a recording device configured to obtain audio and/or visual data when directed toward a womb of a pregnant woman and further configured to transmit the audio and/or visual data; operating a motion processing engine configured to receive the audio and/or visual data, detect motion within the audio and/or visual data, and generate motion data based upon the motion detected within the audio and/or visual data; operating an analytics engine configured to receive the motion data from the motion processing engine, analyze the motion data to determine of the motion data corresponds to a one or more of fetal motion, maternal motion, or other motion, and generate analyzed motion data corresponding to the one or more of fetal motion, maternal motion, or other motion; and operating a data depot configured to receive the analyzed motion data from the analytics engine, store the same as stored data, and transmit the stored data, wherein the stored data is indicative of fetal well-being.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A illustrates components and data within a system configured as a live-recording monitoring system with real-time system output, according to an exemplary embodiment of the present disclosure;

FIG. 1B illustrates components and data within a system, according to an exemplary embodiment of the present disclosure;

FIG. 2 illustrates three different hardware embodiments of a system, according to an exemplary embodiment of the present disclosure;

FIGS. 3A and 3B illustrate systems configured as pre-recording monitoring systems with real-time system output, according to exemplary embodiments of the present disclosure;

FIG. 4 illustrates a system configured for monitoring system replay with data overlay, according to an exemplary embodiment of the present disclosure;

FIG. 5 illustrates a flowchart for using data generated by a system, according to an exemplary embodiment of the present disclosure; and

FIG. 6 shows a block component diagram of elements of devices, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

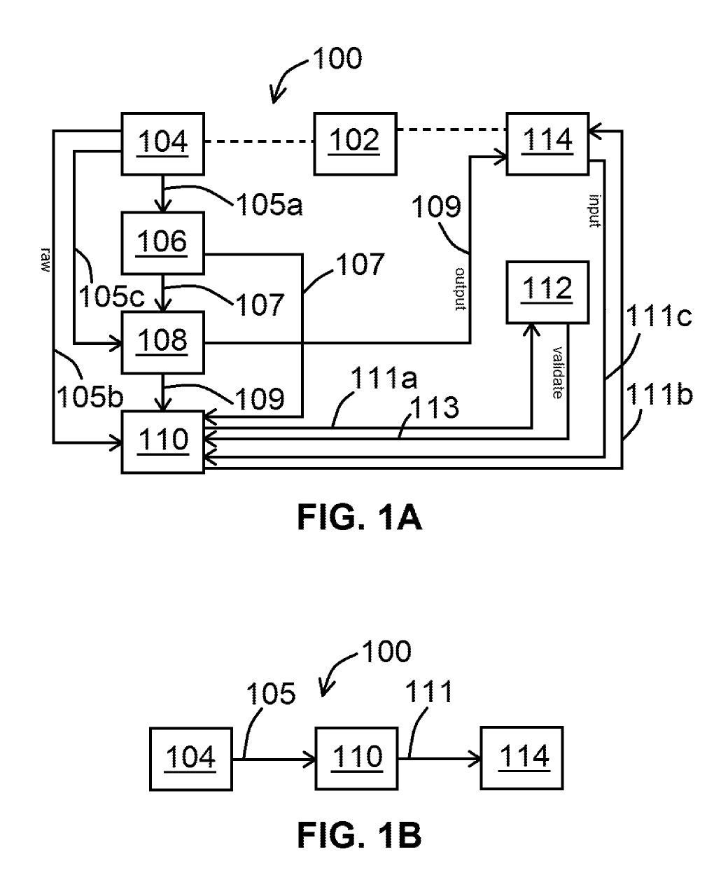

FIG. 1A illustrates an example embodiment of a fetal movement monitoring system 100 of the present disclosure. This exemplary system 100 embodiment illustrates system 100 running in live-recording mode with real-time system output. System 100 shows the user as a pregnant woman 102 whose womb is to be monitored for fetal movement activity. System 100 embodiments comprise an input audio/visual recording device 104 (such as, for example, a video camera and/or a microphone, henceforth embodied as a video camera, for example/illustration) directed at the surface of the pregnant woman's 102 womb (namely at the portion of the pregnant woman's 102 torso where the fetus is present within). As shown in the figure, the audio/visual recording device 104 simultaneously streams its video feed (also referred to as an input audio/visual data stream, shown in FIG. 1A as 105a, 105b, and 105c) to a motion processing engine 106 and to a data depot 110, described in further detail herein, such that input audio/visual data stream 105 (which can also be referred to as 105a, 105b, and/or 105c), as shown in FIG. 1B, can be transmitted from audio/visual recording device 104 to other portions of system 100, such as, for example, motion processing engine 106 and/or data depot 110 (as shown in FIG. 1B), which can be stored as stored data 111 and transmitted out to other portions of system 100, such as user interaction module 114, for example.

Exemplary systems 100 of the present disclosure can use one or more audio/visual recording devices 104, configured as video cameras that obtain video and/or audio, microphones, and the like. Audio/visual recording devices 104 that can only obtain video data would stream its audio/visual data stream 105 only as video. Audio/visual recording devices 104 that can only obtain audio data would stream its audio/visual data stream 105 only as audio. Audio/visual recording devices 104 that can obtain audio and video data would stream its audio/visual data stream 105 as audio and video/visual data.

As noted above, exemplary system 100 embodiments include a motion processing engine 106. Said component processes an input audio/visual data stream 105a frame-by-frame, in at least one embodiment, or as otherwise may be desired. Motion processing engine 106 is configured to detect for any motion, for example, and in particular motion on the surface of the womb.

Exemplary systems 100 of the present disclosure further comprise/include an analytics engine 108. When motion has been detected on the surface of the womb, such as by motion processing engine, analytics engine 108 in distinguishes that movement as fetal movement, maternal movement, or unknown, as analytics engines 108 of the present disclosure are configured to distinguish between or among said movements. Analytics engines 108 are further configured to then generate a representation of the movement (also referred to herein as movement representation(s), which can be or comprise at least part of analyzed motion data 109) and transmit the same along to data depot 110 for logging.

As referenced above, exemplary systems 100 of the present disclosure comprise/include a data depot 110. Data depots 110, in various embodiments, are configured to accept a raw input audio/visual data feed (such as audio/visual data streams 105, 105a, 105b, etc.) from audio/visual recording device 104 and store the same, as may be desired, for later retrieval. Data depots 110, in various embodiments, are also configured to accept movement representation(s) (analyzed motion data 109) from analytics engine 108 and store them for later retrieval, as may be desired. Data depots 100, in various embodiments, are also configured to receive and/or record input from a user interaction module 114 as a record of maternal perception, for example. Data depots 110, in various embodiments, are also configured to store validation data from a validate component 112, as referenced in further detail herein.

Exemplary system 100 embodiments, such as shown in FIG. 1A, may also include/comprise a validate 112 component. Validate component 112, in various embodiments, intercepts system output and user input, and can attempt to measure system correctness/accuracy by comparing movement detected by system 100 to maternally perceived movement, for example.

Various system 100 embodiments can also include/comprise a user interaction module 114 component. User interaction module 114 component acts as an Input and Output interface for the user 102, as referenced in further detail herein. As an output interface, it is configured to display system 100 output, such as representations of system 100 detected movement. As an input interface, for example, it can allow a user 102 to manually record perceptions, such as that of a fetal movement.

In view of the foregoing, and for example, an exemplary audio/visual recording device 104 can record a womb surface, and generate a audio/visual data stream 105, 105a, 105b, and/or 105c (which can contain the same “raw” audio/visual data streaming from audio/visual recording device 104, noting that the differences in reference numbers indicate different paths that the audio/visual data stream 105 can take, namely from audio/visual recording device 104 to any of motion processing engine 106 (via audio/visual data stream 105a), to data depot 110 (via audio/visual data stream 105b), and/or to analytics engine 108 (via audio/visual data stream 105c) containing said recorded information). As shown in FIG. 1A, for example, audio/visual data stream 105a can be transmitted to motion processing engine 106, which is configured to transmit the same, whereby motion processing engine 106 is configured to receive audio/visual data stream 105a and detect motion within audio/visual data stream 105a and ultimately generate motion detection data 107 based upon the detected motion within audio/visual data stream 105a. Motion processing engines 106, in various embodiments, are configured to transmit the motion detection data 107 to analytics engine 108 and/or to data depot 110, which are in turn configured to receive said motion detection data 107 and to analyze the motion detection data 107 and determine of the motions contained therein are fetal motions, maternal motions, and/or other motions, and to generate analyzed motion data 109 based upon the analyzed motion detection data 107, such as in the case of transmitting motion detection data 107 to analytics engine 108, or to store said motion detection data 107 within data depot, such as in the case of transmitting motion detection data 109 to data depot 110. Analytics engines 108, in various embodiments, are configured to transmit the analyzed motion data 109 to data depot 110, which is configured to receive said analyzed motion data 109 from analytics engine 108. Data stored within data depot, whether it be from audio/visual recording device 104, motion processing engine 106, analytics engine 108, validate component 112, and/or user interaction module 114, for example, which can generally be referred to herein as stored data 111. Analytics engines 108, in various embodiments, are also configured to transmit the analyzed motion data 109 to user interaction module 114, such as shown in FIG. 1, which is configured to receive said analyzed motion data 109 from analytics engine 108.

Stored data 111 can be transmitted to validate component 112 as stored data 111a, for example, whereby validate component 112 is configured to receive stored data 111a and to process said stored data 111a in a way to determine whether or not it is accurate and to what extent, if desired, so to generate validated data 113, which can be transmitted back to data depot 110 to be stored itself as stored data 111. Stored data 111 can also be transmitted to user interaction module 114 as stored data 111b, such as to be displayed in one form or another to a user, which can also be transmitted back to data depot 110 as the same stored data 111b or altered stored data 111c, such as in a case where user interaction module modifies stored data 111b in some respect.

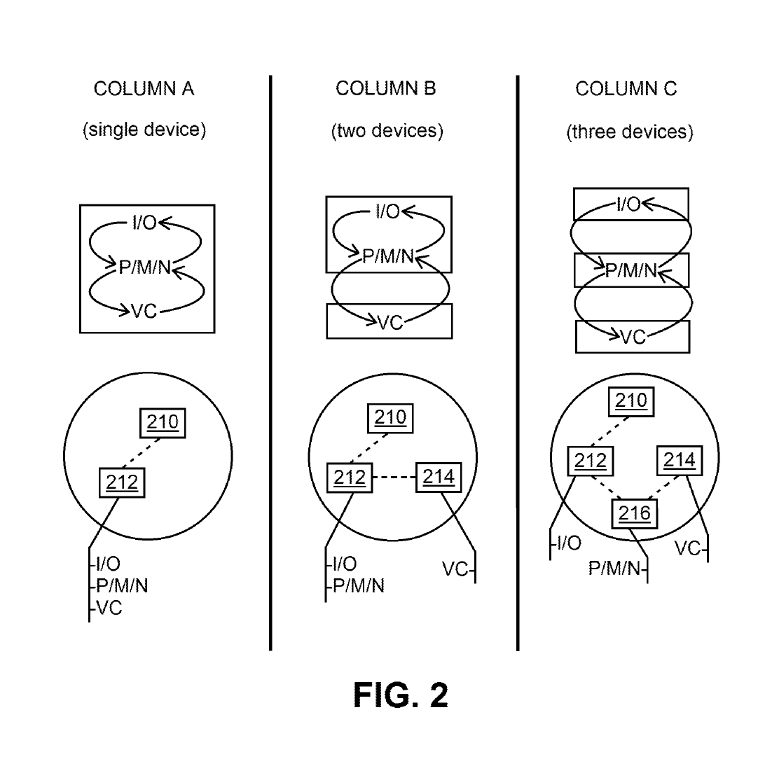

FIG. 2 illustrates 3 non-limiting embodiments of exemplary systems 100 of the present disclosure. The figure shows systems 100 implemented on a single-device, two-devices, and three-devices in columns A, B, and C, respectively. Of course, not all device implementations can be illustrated and other device implementations should be apparent to the skilled artisan from the present disclosure.

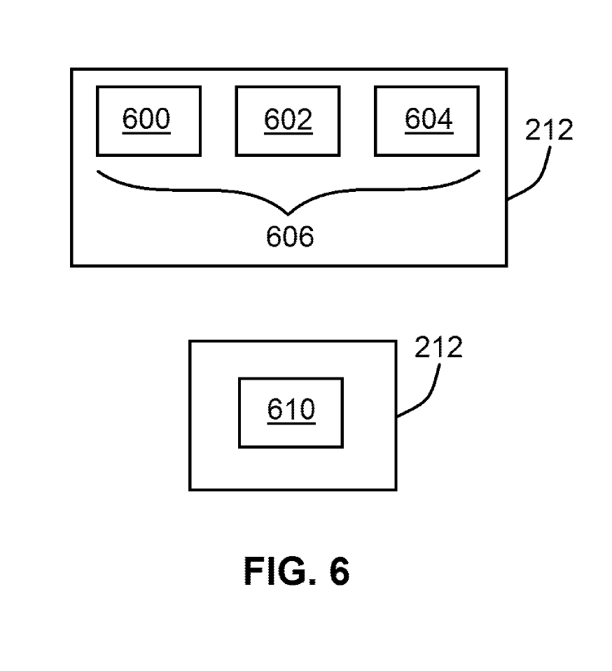

Implementation of various system 100 embodiments includes operation of at least three separate hardware components (I/O, P/M/N, VC), where I/O=Input/Output Interface, P/M/N=Processor/Memory Storage/Network Interface, and VC=Audio/Video Recording Device. These hardware components can reside on the same device or on separate devices, as noted below. P/M/N, as referenced herein, comprises a processor (a computer), memory and/or storage (such as RAM, ROM, a hard drive, flash memory, etc., known and used for data storage), and a network interface configured to connect one or more devices 212, 214, 216 and/or a user interaction module 114 of the present disclosure to one another over a network. As shown in the block component diagram of FIG. 6, an exemplary device of the present disclosure (identified as device 212, but which could be a device 212, device 214, device 216, an audio/visual recording device 104, and/or a user interaction module 114 of the present disclosure, depending on embodiment/configuration) can comprise one or more of a processor 600, memory 602, and/or a network interface 604, identified collectively as P/M/N 606. The same or different of the present disclosure (identified as device 212, but which could be a device 212, device 214, device 216, an audio/visual recording device 104, and/or a user interaction module 114 of the present disclosure, depending on embodiment/configuration) comprises an input/output interface 610. VC, as referenced herein, comprises or is part of an audio/visual recording device 104 and/or one or more of a device 212, device 214, device 216, and/or a user interaction module 114 of the present disclosure. Memory 602, as referenced herein, can comprise or be part of a data depot 110.

In a first embodiment, shown in column A of FIG. 2, system 100 is implemented on a single device 212. Device 212, in at least one embodiment, comprises a smartphone or similar device 212. In each exemplary embodiment shown in FIG. 2, for example, devices 212 can also be a tablet computer, laptop computer, desktop computer, or other computing device. In the embodiment shown in column A of FIG. 2, the smartphone device 212 contains all three hardware components (I/O, P/M/N, VC) noted above that are used to operate system 100. User 210, as shown in FIG. 2, represents the pregnant woman 102 being monitored, but could also be a different user, such as a friend, family member, medical person, and the like.

In the second embodiment, shown in column B of FIG. 2, system 100 is implemented on two devices 212, 214. Device 212, in at least one embodiment, comprises a smartphone or similar device 212, such as used in column A of FIG. 2. Device 214, as shown in column B of FIG. 2, is a audio/visual recording device (such as, for example, audio/visual recording device 104) that transmits its audio/visual data feed (input audio/visual data stream 105) to device 212. Device 214 can also be a separate smartphone (similar or the same type of device used as device 212), utilizing only its audio/visual recording device feature, or the audio/visual recording device feature and not other features used within device 212, for example. Having the audio/visual recording device 104 separate from the Input/Output interface, for example, allows the user better flexibility in viewing the interface without interfering with the audio/visual recording device 104 view angle.

In the third embodiment, shown in column C of FIG. 2, system 100 is implemented on three devices 212, 214, 216. Device 212, in at least one embodiment, comprises a smartphone or similar device 212, such as used in column A of FIG. 2. Device 214, as shown in column C of FIG. 2, is a video camera (such as, for example, audio/visual recording device 104) that transmits its audio/visual data feed (input audio/visual data stream 105) to device 212. Device 214 can also be a separate smartphone (similar or the same type of device used as device 212), utilizing only its audio/visual recording device feature, or the audio/visual recording device feature and not other features used within device 212, for example. Device 216, as referenced in this exemplary embodiment, is manifest as a workstation server used for Processing/Memory Storage/Networking Communication. Device 216 can also be any general type of computer, such as a laptop computer, desktop computer, tablet computer, etc., or even be yet another smartphone (similar or the same type of device used as device 212), using its P/M/N features.

In view of the foregoing, exemplary systems 100 of the present disclosure can use any number of devices 212, 214, 216, etc., which can individually or collectively perform each of the I/O, P/M/N, and VC functions. Column A of FIG. 2 shows one device 212 that performs each function. Column B of FIG. 2 shows a device 212 that performs the I/O and P/M/N functions, and a separate device 214 that performs the VC function. Column C of FIG. 2 shows three devices 212, 214, and 216, with each performing one of the aforementioned functions. In view of the foregoing, motion processing engine 106, analytics engine 108, validate component 112, and/or user interaction module 114 may partially and/or fully comprise software, whereby said software is configured for storage upon the memory storage portion of P/M/N, and operable using the processing portion (the processor/computer) of P/M/N, of one or more of devices 212, 214, and/or 216 referenced herein.

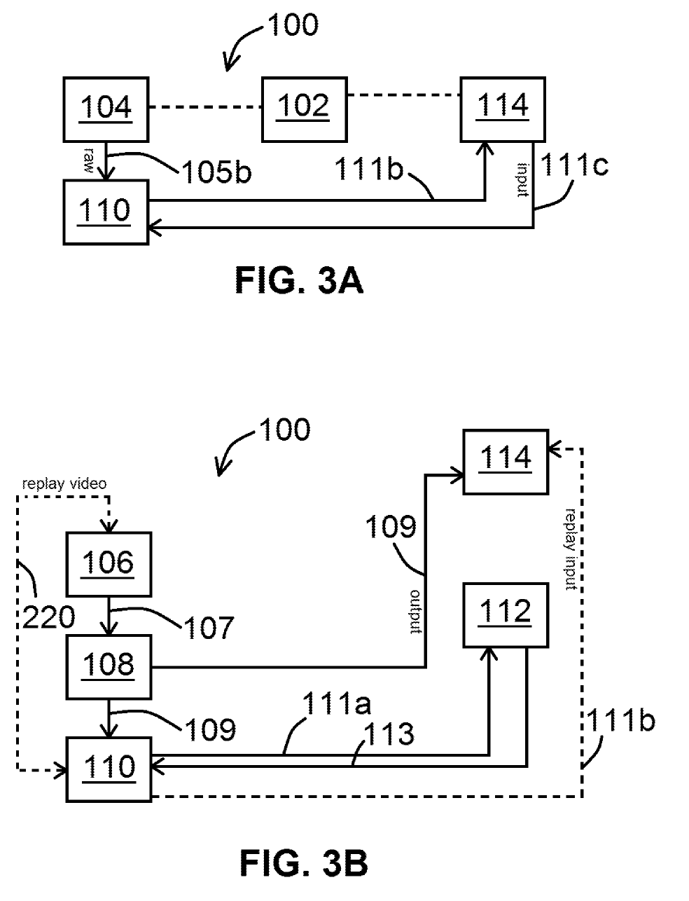

FIGS. 3A and 3B illustrate various exemplary embodiments of fetal movement monitoring systems 100 of the present disclosure. System 100, as shown in FIG. 3A, is shown as running in a pre-recording mode with real-time system output. The components of this figure overlap with those shown in FIG. 1A, but the difference with this embodiment, however, is that the input audio/visual data feed (input audio/visual data stream 105) is not processed by the system as it is being recorded, such as being processed with one or both of a motion processing engine 106 and/or an analytics engine 108 shown in FIG. 1A. Rather, input audio/visual data stream 105, as shown in FIG. 3A, is post-processed by system 100 as a single input audio/visual data file, for example, or a series of individual audio/visual data files. FIG. 3A illustrates the input audio/visual data recording of the surface of a womb of a pregnant woman 102. This audio/visual data input stream, along with synchronized maternal perception input collected through a user interaction module 114 for example, is stored at data depot 110 for later processing by system 100. Afterwards, as shown in FIG. 3B, pre-recorded data can be processed by system 100. Analogous to how system 100 processes data represented in FIG. 1A, system 100, as shown in FIG. 3B, imitates that it is running in a live-recording mode, by replaying the saved input video (replayed audio/visual data stream 220, versus any of input audio/visual data streams 105, 105a, or 105b) and streaming it from data depot 110 to motion processing engine 106. It also mimics receiving input maternal perception (if this data is available), by replaying it on the user interaction module 114. Replayed audio/visual data stream 220 could be processed the same or similar to how input audio/visual data stream 105a could be processed by motion processing engine 106, such as shown in FIG. 1, to generate motion detection data 107 from the replayed audio/visual data stream 220, which could be transmitted to analytics engine 108, analyzed as referenced herein to generate analyzed motion data 109, which could then be transmitted to data depot 110 as referenced herein.

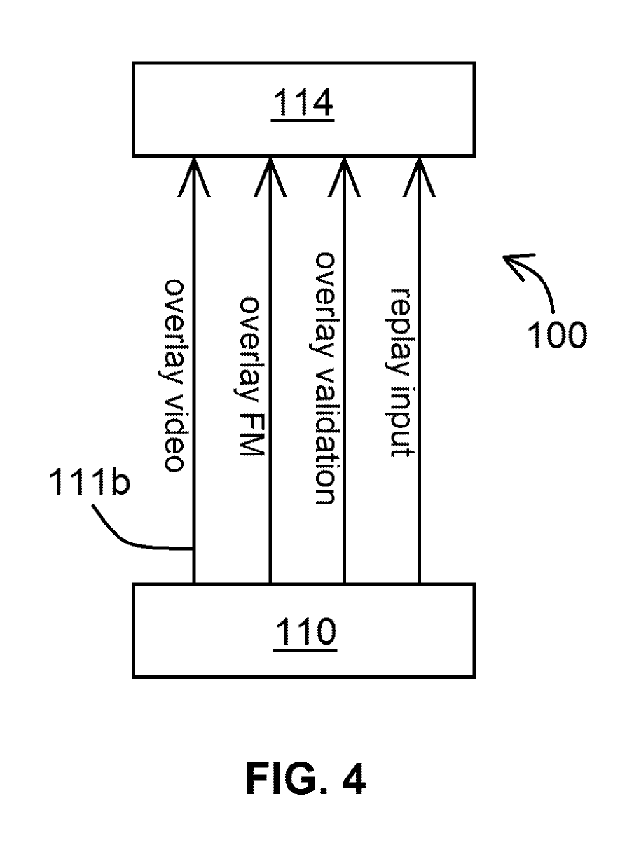

FIG. 4 illustrates a replay of an exemplary system 100 of the present disclosure simulating as if the user (such as a pregnant woman 102 or another user) were running it in live-recording mode with real-time system output. Data is simply retrieved from the data depot 110, and streamed onto the user interaction module 114. This system 100 replay is useful for reviewing a past monitoring session. This system 100 replay is also useful for seeing a full rendering of the system 100 output when available computing resources are insufficient to process the input audio/visual data stream in real-time, for example.

The various systems 100 herein can be used to determine fetal well-being such as by way of obtaining raw data using a audio/visual recording device (input audio/visual data stream 105, 105a, 105b, and/or 105c), generating motion detection data 107, generating analyzed motion data 109, and/or generating validated data 113, which can be displayed in user interaction module 114 or otherwise be made available to a user of system 100 (or portions thereof). Said data can identify movements that are attributed to the fetus and not attributed to the mother or other movement, and said fetal movement data can be analyzed and/or displayed, and potentially compared to benchmarks relating to fetal movement or lack thereof, to determine fetal well-being. For example, if certain benchmarks identify frequency and/or extent/strength of fetal movement, and data obtained from system 100 identifies fetal movement frequency and/or extent/strength that meet said benchmarks, then a determination could be made that based upon said data from system 100 that the fetus makes appropriate movements. Conversely, if data obtained from system 100 identifies fetal movement frequency and/or extent/strength that does not meet said benchmarks, such as less frequent movement and/or weaker movements, then a determination could be made that based upon said data from system 100 that the fetus may have a compromised well-being. Furthermore, should benchmarks identifying frequency and/or extent/strength of fetal movement as being related to one or more fetal conditions be met by data obtained by system 100, diagnoses of one or more fetal conditions could be made based upon said data, and a treatment plan could be generated/determined based upon said diagnoses.

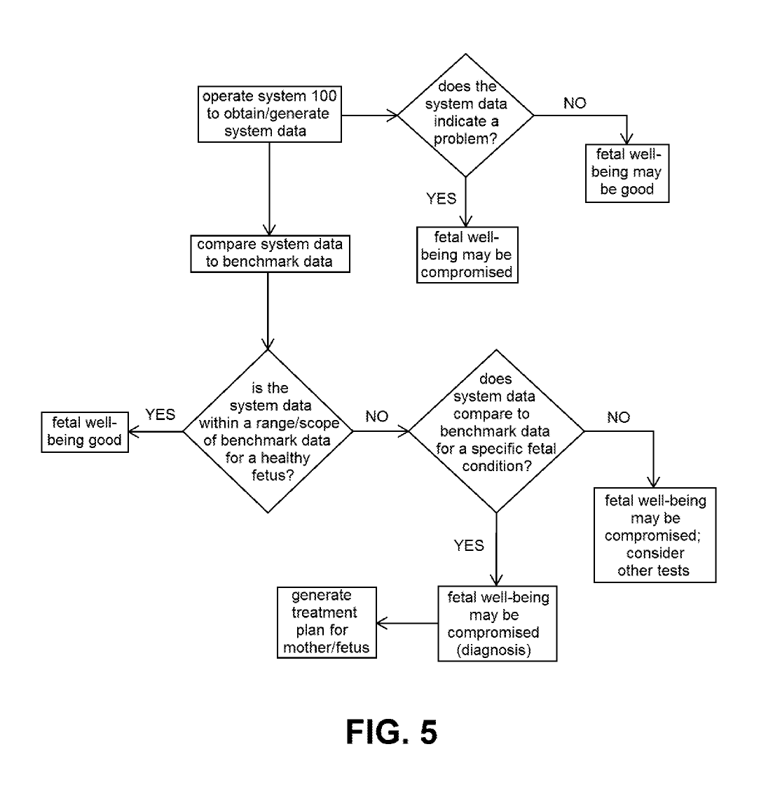

FIG. 5 shows a flowchart consistent with the foregoing, whereby system 100 is operated to generate system data, and whereby the system data can be compared to benchmark data or generally used to identify a particular problem/condition with the mother and/or the fetus. If the system data identifies a problem, fetal well-being could be compromised, and if no problem is identified, then fetal well-being may be okay/good, noting that other conditions may exist that are not attributed to fetal movement. If the system data is compared to benchmark data, a determination as to whether the system data is within a range/scope of said benchmark data for a healthy fetus, and if so, then fetal well-being may be okay/good, again noting that other conditions may exist that are not attributed to fetal movement, and if not, the system data could be compared to data indicative of one or more fetal conditions. Should the system data compare/align with the data indicative of one or more fetal conditions, then fetal well-being could be compromised, with a potential diagnosis of one or more said conditions being made, and a treatment plan could be generated and potentially executed to treat the mother and/or fetus. Should the system data not compare/align with the data indicative of one or more fetal conditions, but is still within a range of an unhealthy fetus, then fetal well-being could still be compromised, and potential other tests known or developed in the art could be considered and/or performed.

While various embodiments of systems and devices for identifying fetal movements in a audio/visual data feed and using the same to assess fetal well-being and other methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A system, comprising:
a recording device configured to obtain audio and/or visual data when directed toward a womb of a pregnant woman and further configured to transmit the audio and/or visual data;
a motion processing engine configured to receive the audio and/or visual data, detect motion within the audio and/or visual data, and generate motion data based upon the motion detected within the audio and/or visual data;
an analytics engine configured to receive the motion data from the motion processing engine, analyze the motion data to determine of the motion data corresponds to a one or more of fetal motion, maternal motion, or other motion, and generate analyzed motion data corresponding to the one or more of fetal motion, maternal motion, or other motion; and
a data depot configured to receive the analyzed motion data from the analytics engine, store the same as stored data, and transmit the stored data.

2. The system of claim 1, wherein the analytics engine is further configured to receive the audio and/or visual data from the recording device.

3. The system of claim 1, wherein the data depot is further configured to receive the audio and/or visual data from the recording device and to store the same as stored data.

4. The system of claim 1, further comprising:
a user interaction module configured to receive analyzed motion data from the analytics engine, to receive maternal perception input data synchronized with the audio and/or visual data, and to display at least one of the analyzed motion data and/or the maternal perception input data.

5. The system of claim 4, wherein the user interaction module is an input/output device.

6. The system of claim 4, further comprising:
a validate component configured to receive the stored data from the data depot, compare the stored data with the maternal perception input data to generate validated data, and to transmit the validated data to the data depot to be stored as additional stored data.

7. The system of claim 1, configured for operation upon a single device, the single device comprising:
an input/output interface;
a processor/memory storage/network interface; and
the recording device.

8. The system of claim 7, wherein the single device is selected from the group consisting of a smartphone, a tablet computer, and a laptop computer.

9. The system of claim 1, configured for operation upon a first device and a second device, the first device comprising an input/output interface and a processor/memory storage/network interface, and the second device comprising the recording device, wherein the first device is configured to receive the audio and/or visual data from the second device.

10. The system of claim 9, wherein the first device is selected from the group consisting of a smartphone, a tablet computer, and a laptop computer.

11. The system of claim 1, configured for operation upon a first device, a second device, and a third device, the first device comprising an input/output interface, the second device comprising the recording device, and the third device comprising a processor/memory storage/network interface.

12. The system of claim 11, wherein the third device is configured to receive the audio and/or visual data from the second device, store the audio and/or visual data as the stored data, and transmit the stored data to the first device.

13. The system of claim 11, wherein the first device comprises a smartphone or a tablet, and wherein the third device comprises a laptop computer or a desktop computer.

14. The system of claim 1, wherein the stored data is indicative of fetal well-being.

15. The system of claim 1, wherein the stored data is indicative of a diagnosis of a fetal condition.

16. A system, comprising:
a recording device configured to obtain audio and/or visual data when directed toward a womb of a pregnant woman and further configured to transmit the audio and/or visual data;
a motion processing engine configured to receive the audio and/or visual data, detect motion within the audio and/or visual data, and generate motion data based upon the motion detected within the audio and/or visual data;
an analytics engine configured to receive the motion data from the motion processing engine, analyze the motion data to determine of the motion data corresponds to a one or more of fetal motion, maternal motion, or other motion, and generate analyzed motion data corresponding to the one or more of fetal motion, maternal motion, or other motion;
a data depot configured to receive the analyzed motion data from the analytics engine, store the same as stored data, and transmit the stored data; and
a validate component configured to receive the stored data from the data depot, compare the stored data with maternal perception input data obtained by a user interaction module to generate validated data, and to transmit the validated data to the data depot to be stored as additional stored data.

17. The system of claim 16, configured for operation upon a single device, the single device comprising:
an input/output interface;
a processor/memory storage/network interface; and
the recording device;
wherein the single device is selected from the group consisting of a smartphone, a tablet computer, and a laptop computer.

18. The system of claim 16, configured for operation upon a first device and a second device, the first device comprising an input/output interface and a processor/memory storage/network interface, and the second device comprising the recording device, wherein the first device is configured to receive the audio and/or visual data from the second device, and wherein the first device is selected from the group consisting of a smartphone, a tablet computer, and a laptop computer.

19. The system of claim 16, configured for operation upon a first device, a second device, and a third device, the first device comprising an input/output interface, the second device comprising the recording device, and the third device comprising a processor/memory storage/network interface, wherein the third device is configured to receive the audio and/or visual data from the second device, store the audio and/or visual data as the stored data, and transmit the stored data to the first device, and wherein the first device comprises a smartphone or a tablet, and wherein the third device comprises a laptop computer or a desktop computer.

20. A method of determining fetal well-being, comprising the steps of:
operating a recording device configured to obtain audio and/or visual data when directed toward a womb of a pregnant woman and further configured to transmit the audio and/or visual data;
operating a motion processing engine configured to receive the audio and/or visual data, detect motion within the audio and/or visual data, and generate motion data based upon the motion detected within the audio and/or visual data;
operating an analytics engine configured to receive the motion data from the motion processing engine, analyze the motion data to determine of the motion data corresponds to a one or more of fetal motion, maternal motion, or other motion, and generate analyzed motion data corresponding to the one or more of fetal motion, maternal motion, or other motion; and
operating a data depot configured to receive the analyzed motion data from the analytics engine, store the same as stored data, and transmit the stored data, wherein the stored data is indicative of fetal well-being.
